(19) United States
(12) Patent Application Publication
(10) Pub. No.: US 2025/0140350 A1
(43) Pub. Date: May 1, 2025

(54) METHODS AND SYSTEMS FOR IMPROVED BASE CALL RESOLUTION OF ELECTROPHEROGRAM OUTPUT GENERATED FROM MIXED SAMPLES

(71) Applicant: Pathogenomix, Inc., Santa Cruz, CA (US)

(72) Inventor: Oystein Friestad Saebo, Santa Cruz, CA (US)

(21) Appl. No.: 18/930,599

(22) Filed: Oct. 29, 2024

Related U.S. Application Data
(60) Provisional application No. 63/594,321, filed on Oct. 30, 2023.

(51) Int. Cl. G16B 30/20; G16B 40/10; G16B 40/20; G16B 50/30

(52) U.S. Cl. CPC G16B 30/20; G16B 40/10; G16B 40/20; G16B 50/30

(57) ABSTRACT

Systems and methods are disclosed for identifying multiple species in mixed samples using Sanger sequencing. The systems and methods receive a chromatogram from an electropherogram device and generate a plurality of base positions along the chromatogram's time axis. Each base position is generated at either a peak or inflection point in the chromatogram, and multiple base positions are generated at each wide peak in the chromatogram. The systems and methods call a base at the base position. Once bases are called, the systems and methods generate a base calling array that stores called bases in a plurality of indexes. Each index in the plurality of indexes corresponds to a window along the time axis and called bases within the window are added to the corresponding index. Each window partially overlaps with another window, and called bases at base positions that overlap two windows are added to both indexes.

### CROSS-REFERENCE TO RELATED APPLICATIONS

This application claims priority to U.S. Provisional Patent Application No. 63/594,321, filed Oct. 30, 2023, the entire disclosure of which is incorporated herein by reference in its entirety, for any and all purposes.

### BACKGROUND

Sanger sequencing of clinical samples can provide accurate identification of pathogens present in a sample. However, conventional Sanger sequencing typically requires a pure isolate as the source material for sequence runs. If mixed samples are sequenced, conventional Sanger sequencers may produce a convoluted chromatogram that is difficult to interpret and typically discarded.

### SUMMARY

Described herein are systems and methods for identifying multiple species in mixed samples using Sanger sequencing. The systems and methods may detect multiple species with identical bases at the same sequencing position by analyzing, among other things, shoulders, inflection points, and wide peaks in electropherograms. The systems and methods described herein may automatically detect multiple species from a sequence run of a clinical sample containing multiple species, without needing to isolate the different species for separate sequence runs.

Existing Sanger sequencing technology is only capable of sequencing a pure isolate as the source material, and chromatograms of mixed samples are typically discarded. However, where isolates are required for sequencing, isolates may be prepared using cell culture, which may take several days or weeks, and has a failure rate of about 30% to about 50%. This processing time and relatively high failure rate can result in significant delays in Sanger sequence processing, up to days or weeks for the identification of the sample.

To address the deficiencies of conventional approaches to Sanger sequencing, the systems and methods described herein can automatically determine multiple species from a sequence run of a clinical sample containing multiple species by detecting peak shoulders, inflection points, wide peaks, and irregularly spaced peaks, among other aspects, in the Sanger sequencing chromatogram. Unlike conventional Sanger sequencing approaches, which typically discard chromatograms indicating such features, the techniques described herein can be used to accurately and efficiently process these chromatograms to detect multiple species with the same base at the same base position. In doing so, the systems and methods described herein may efficiently detect and identify multiple species from a sequencing run, thereby improving sequencing efficiency and accuracy. These and other improvements are described in further detail herein.

At least one aspect of the present disclosure is directed to a system. The system can include one or more processors coupled to memory. The processors are configured to receive a chromatogram from an electropherogram device. The chromatogram has multiple signals with a time axis and an intensity axis. The processors are configured to generate, from the chromatogram, a plurality of base positions along the time axis, wherein each base position is generated at either a peak or inflection point in the chromatogram, and wherein multiple base positions are generated at each wide peak in the chromatogram. Each wide peak has a full width half max (FWHM) greater than a predetermined width. The processors are configured to call, for each base position corresponding to the chromatogram, a base at the base position corresponding to a signal of the multiple signals of the chromatogram, The processors are configured to generate a base calling array that stores called bases. The base calling array includes a plurality of indexes, each index in the plurality of indexes corresponds to a window along the time axis such that called bases within the window are added to the corresponding index. Each window partially overlaps with another window, and called bases at base positions that overlap two windows are added to both indexes.

In some implementations, to generate the base calling array, the one or more processors may be further configured to identify a first anchor peak and use the first anchor peak position along the time axis as a first index in the plurality of indexes. To generate the base calling array, the one or more processors may be further configured to, starting at a second index after the first index, for each window, identify an anchor peak if present. If the anchor peak is present, the one or more processors may be further configured to adjust the window by a distance so that a midpoint of the anchor peak is a midpoint of the window. The one or more processors may be further configured to adjust subsequent windows by the same distance. In some implementations, the predetermined width is greater than about 1.5X, where X is a FWHM of an anchor peak. Generating multiple base positions at each wide peak in the chromatogram may include generating a base position at a first predetermined position left of each wide peak and a second predetermined position right of each wide peak. Generating multiple base positions at each wide peak in the chromatogram may include identifying whether each wide peak skews left, right, or is centered. For each wide peak that skews left, the one or more processors may be configured to generate base positions at (⅚)X left of each wide peak and (⅙)X right of each wide peak. For each wide peak that skews right, the one or more processors may be configured to generate base positions at (⅚)X right of each wide peak and (⅙)X left of each wide peak. For each wide peak that is centered, the one or more processors may be configured to generate base positions at ( 5/12)X right and left of each wide peak.

In some implementations, the one or more processors may be further configured to determine a number of indexes that include multiple bases and cause a client device to present a notification responsive to determining that the indexes with multiple bases exceeds a threshold value. Determining the number of indexes that include the multiple bases may include determining the number of indexes including three bases and the number of indexes including four bases. The threshold value may include different threshold values for the number of indexes including the three bases and the number of indexes including the four bases.

In some implementations, the one or more processors may be further configured to generate a list of species from a reference database that have a DNA sequence that at least partially matches a portion of the base calling array. The one or more processors may group the list of species according to Levenshtein distances between respective DNA sequences. Grouping the list of species according to Levenshtein distances may include grouping species together that have a percentage Levenshtein distance of 5% or less.

In some implementations, the one or more processors may be further configured to cause a client device to present a notification responsive to determining that more than a threshold number of groups are generated. In some implementations, the threshold number of groups is 3 groups. A signal of the multiple signals of the chromatogram may correspond to a base that is one of A, C, T, or G.

At least one aspect of the present disclosure is directed to a method. The method can include receiving a chromatogram from an electropherogram device. The chromatogram has multiple signals with a time axis and an intensity axis. The method can include generating, from the chromatogram, a plurality of base positions along the time axis, wherein each base position is generated at either a peak or inflection point in the chromatogram, and wherein multiple base positions are generated at each wide peak in the chromatogram. Each wide peak has a full width half max (FWHM) greater than a predetermined width. The method can include calling, for each base position corresponding to the chromatogram, a base at the base position corresponding to a signal of the multiple signals of the chromatogram. The method can include generating a base calling array that stores called bases. The base calling array includes a plurality of indexes, each index in the plurality of indexes corresponds to a window along the time axis such that called bases within the window are added to the corresponding index, wherein each window partially overlaps with another window, and called bases generated at base positions that overlap two windows are added to both indexes.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification. Aspects can be combined and it will be readily appreciated that features described in the context of one aspect of the present disclosure can be combined with other aspects. Aspects can be implemented in any convenient form. For example, by appropriate computer programs, which may be carried on appropriate carrier media (computer readable media), which may be tangible carrier media (e.g. disks) or intangible carrier media (e.g. communications signals). Aspects may also be implemented using suitable apparatus, which may take the form of programmable computers running computer programs arranged to implement the aspect. As used in the specification and in the claims, the singular form of “a,” “an,” and “the” include plural referents unless the context clearly dictates otherwise.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1 illustrates an example system for identifying multiple species in chromatograms of mixed samples, in accordance with one or more implementations.

FIG. 2 illustrates an example dataflow diagram of a process for identifying multiple species in chromatograms of mixed samples, in accordance with one or more implementations.

FIG. 3 illustrates a flow chart of an example method for identifying multiple species in chromatograms of mixed samples, in accordance with one or more implementations.

FIG. 4 illustrates a flow chart of an example method for notifying a client device if a base calling array includes too many indexes with multiple bases, in accordance with one or more implementations.

FIG. 5 illustrates a flow chart of an example method for analyzing base calling arrays, in accordance with one or more implementations.

FIG. 6 illustrates a block diagram of an example computing system suitable for use in the various arrangements described herein, in accordance with one or more implementations.

### DETAILED DESCRIPTION

Below are detailed descriptions of various concepts related to and implementations of techniques, approaches, methods, apparatuses, and systems for removing data from strings. The various concepts introduced above and discussed in detail below may be implemented in any of numerous ways, as the described concepts are not limited to any particular manner of implementation. Examples of specific implementations and applications are provided primarily for illustrative purposes.

As used herein, the term “anchor point” means a single peak in a chromatogram corresponding to more than one species (e.g., multiple bacteria species) that have identical bases in the same position along the time axis of the chromatogram. The fused fluorescent signal representing a single peak is a compromise between the contributing signals of the different species having the same bases. Anchor peaks may be used as the center points of indexes when generating base calling arrays. This may be useful because fluorescent peaks may be displaced in a sample with multiple organisms (e.g., because of the difference in migration speed between organisms in gel electrophoresis).

### Improving Multiple Base Detection of Multiple Species at the Same Position.

Existing Sanger sequencing techniques typically require a pure isolate source material for the sequence run. Most conventional Sanger sequencers come with a warning that operators must provide a pure isolate as the source material for the sequence run. If mixed samples are sequenced, sequencers may produce a convoluted chromatogram that includes overlapping peaks that manifest as, among other things, peak shoulders, inflection points, wide peaks, and irregularly spaced peaks. These convoluted chromatograms are typically discarded as poor-quality data.

Discarding results from mixed samples can decrease sampling efficiency and increase the time to identify a sample. Using pure isolate samples typically means culturing the sample, but colonies can grow slowly. Typically, colonies are cultured for two to four days, though some organisms may be cultured for weeks to grow enough material for sampling. However, culturing has a high failure rate, where additional organisms may contaminate the cultures. If Sanger sequencing is used to identify a patient's bacterial infection for effective treatment, the additional delay needed to prepare a pure isolate sample can have disastrous consequences.

To address these and other issues, the systems and methods described herein can automatically identify multiple species in mixed samples using Sanger sequencing chromatograms. The systems and methods may detect identical bases from multiple species at the same sequencing position by detecting, among other things, peak shoulders, inflection points, and wide peaks in chromatograms (also called electropherograms). In doing so, the techniques described herein can be used to improve efficiency and accuracy of identification of multiple species in chromatogram data, thereby decreasing overall analysis time and increasing productivity. These techniques increase the number of species that can be identified in a single sequencing run and improve data resolution.

One example use case for the present techniques includes identifying species in a sample from a polymicrobial infection. Since multiple species may be identified with the present techniques, the sample may not require culturing before sampling. The polymicrobial infection may include a mix of gram positive and gram negative bacteria. For example, the sample may include one or more gram positive bacteria, including bacteria from the genera Bacillus, Enterococcus, Leuconostoc, Listeria, Staphylococcus, and/or Streptococcus. For example, the gram-positive bacteria may include one or more actinomycetota, including mycobacteria and Nocardia. For example, the sample may include one or more gram negative bacteria, including bacteria from the genera Yokenella, Salmonella, Erwinia, Curtobacterium, Shigella, Raoultella, Pseudomonas, Providencia, Proteus, Pluralibacter, Plesiomonas, Pantoea, Neisseria, Morganella, Lelliottia, Legionella, Leclercia, Kosakonia, Klebsiella, Hafnia, Escherichia, Enterobacter, Elizabethkingia, Cronobacter, Citrobacter, Burkholderia, and/or Acinetobacter. The polymicrobial infection may include one or more fungi (e.g., as sequenced with the ITS2 gene).

For example, the sample may include one or more gram positive bacteria, including Bacillus anthracis, Bacillus cereus, Enterococcus durans, Enterococcus faecalis, Enterococcus faecium, Enterococcus gallinarum, Enterococcus hirae, Enterococcus mundtii, Leuconostoc citreum, Leuconostoc inhae, Leuconostoc mesenteroides, Leuconostoc palmae, Leuconostoc pseudomesenteroides, Listeria innocua, Listeria monocytogenes, Staphylococcus aureus, Staphylococcus capitis, Staphylococcus caprae, Staphylococcus chromogenes, Staphylococcus delphini, Staphylococcus epidermidis, Staphylococcus gallinarum, Staphylococcus haemolyticus, Staphylococcus hyicus, Staphylococcus lugdunensis, Staphylococcus pasteuri, Staphylococcus pseudintermedius, Staphylococcus warneri, Staphylococcus xylosus, Streptococcus agalactiae, Streptococcus dysgalactiae, Streptococcus gallolyticus, Streptococcus infantis, Streptococcus intermedius, Streptococcus macedonicus, Streptococcus mutans, Streptococcus oralis, Streptococcus orisratti, Streptococcus peroris, Streptococcus pyogenes, Streptococcus salivarius, Streptococcus thermophilus, Streptococcus vestibularis, Enterococcus malodoratus, Staphylococcus carnosus, Staphylococcus devriesci, Streptococcus equinus, Staphylococcus schleiferi, and/or Bacillus anthracis.

For example, the sample may include one or more gram negative bacteria, including Yokenella regensburgei, Salmonella enterica, Erwinia mediterraneensis, Curtobacterium plantarum, Shigella sonnei, Shigella flexneri, Shigella dysenteriae, Shigella boydii, Salmonella bongori, Raoultella terrigena, Raoultella planticola, Raoultella ornithinolytica, Raoultella electrica, Pseudomonas veronii, Pseudomonas putida, Providencia vermicola, Providencia rustigianii, Providencia rettgeri, Providencia huaxiensis, Providencia alcalifaciens, Proteus mirabilis, Pluralibacter gergoviae, Plesiomonas shigelloides, Pantoea wallisii, Pantoea vagans, Pantoea stewartia, Pantoea rwandensis, Pantoea pleuroti, Pantoea eucrina, Pantoea eucalypti, Pantoea endophytica, Pantoea dispersa, Pantoea deleyi, Pantoea cypripedii, Pantoea conspicua, Pantoea coffeiphila, Pantoea brenneri, Pantoea anthophila, Pantoea ananatis, Pantoea allii, Pantoea alhagi, Pantoea agglomerans, Neisseria meningitidis, Morganella morganii, Lelliottia amnigena, Legionella pneumophila, Leclercia adecarboxylata, Kosakonia cowanii, Klebsiella variicola, Klebsiella spallanzanii, Klebsiella quasivariicola, Klebsiella quasipneumoniae, Klebsiella pasteurii, Klebsiella oxytoca, Klebsiella michiganensis, Klebsiella huaxiensis, Klebsiella grimontii, Klebsiella Africana, Klebsiella aerogenes, Hafnia paralvei, Hafnia alvei, Escherichia whittamii, Escherichia ruysiae, Escherichia fergusonii, Escherichia coli, Escherichia albertii, Enterobacter wuhouensis, Enterobacter vonholyi, Enterobacter soli, Enterobacter sichuanensis, Enterobacter roggenkampii, Enterobacter quasiroggenkampii, Enterobacter quasimori, Enterobacter quasihormacchei, Enterobacter oligotrophicus, Enterobacter mori, Enterobacter ludwigii, Enterobacter kobei, Enterobacter huaxiensis, Enterobacter hormacchei, Enterobacter dykesii, Enterobacter cloacac, Enterobacter chuandaensis, Enterobacter cancerogenus, Enterobacter bugandensis, Enterobacter asburiae, Elizabethkingia anopheles, Cronobacter turicensis, Cronobacter sakazakii, Cronobacter malonaticus, Cronobacter dublinensis, Citrobacter youngae, Citrobacter werkmanii, Citrobacter tructae, Citrobacter telavivensis, Citrobacter sedlakii, Citrobacter rodentium, Citrobacter portucalensis, Citrobacter pasteurii, Citrobacter koseri, Citrobacter gillenii, Citrobacter freundii, Citrobacter farmer, Citrobacter europacus, Citrobacter cronac, Citrobacter braakii, Citrobacter amalonaticus, Burkholderia vietnamensis, Burkholderia multivorans, Burkholderia dolosa, Burkholderia cepacian, Burkholderia cenocepacia, Acinetobacter ursingii, Acinetobacter soli, Acinetobacter seifertii, Acinetobacter pittii, Acinctobacter nosocomialis, Acinetobacter lactucae, Acinetobacter junii, Acinetobacter haemolyticus, Acinetobacter colistiniresistens, Acinetobacter calcoaceticus, and/or Acinetobacter baumannii.

For example, the sample may include one or more actinomycetota, including Mycobacterium tuberculosis, Mycobacterium kyorinense, Mycobacterium intracellulare, Mycobacterium shigaense, Mycobacterium kansasii, Mycobacterium paragordonac, Mycobacterium celatum, Mycobacterium saskatchewanense, Mycobacterium mantenii, Mycobacterium seoulense, Mycobacterium malmoense, Mycobacterium vicinigordonae, Mycobacterium grossiae, Mycobacterium paraterrae, Mycobacterium sp. ‘sulfur cave’, Mycobacterium novum, Mycobacterium haemophilus, Mycobacterium noviomagense, Nocardia wallacei, Mycobacterium marinum, Mycobacterium shottsii, Mycobacterium spongiac, Mycobacterium basiliense, Mycobacterium kubicac, Mycobacterium leprae, Mycobacterium senriense, Mycobacterium heckeshornense, Mycobacterium paraintracellulare, Mycobacterium avium, Mycobacterium lepromatosis, Mycobacterium florentinum, Mycobacterium ulcerans, Mycobacterium pseudoshottsii, Mycobacterium frederiksbergense, Mycobacterium lacus, Mycobacterium marseillense, Mycobacterium stomatepiae, Mycobacterium simiae, Mycobacterium dioxanotrophicus, Mycobacterium canettii, Mycobacterium shinjukuense, Mycobacterium neumannii, Mycobacterium barrassiae, Mycobacterium orygis, Mycobacterium conspicuum, Mycobacterium bohemicum, Mycobacterium interjectum, Mycobacterium paraense, Mycobacterium parascoulense, Mycobacterium paraffinicum, Mycobacterium europaeum, Mycobacterium scrofulaceum, Mycobacterium palustre, Mycobacterium nebraskense, Mycobacterium montefiorense, Mycobacterium szulgai, Mycobacterium pseudokansasii, Mycobacterium innocens, Mycobacterium persicum, Mycobacterium attenuatum, Mycobacterium gastri, Mycobacterium bouchedurhonense, Mycobacterium hackensackense, Mycobacterium helveticum, Mycobacterium palauense, Mycobacterium aquaticum, Mycobacterium mungi, Mycobacterium asiaticum, Mycobacterium intermedium, Mycobacterium kyogaense, Mycobacterium tilburgii, Mycobacterium lentiflavum, Mycobacterium ulcerans, Mycobacterium sherrisii, Mycobacterium ahvazicum, Nocardia gamkensis, Nocardia Africana, Nocardia vermiculata, Mycobacterium alsense, Mycobacterium parmense, Mycobacterium fragae, Mycobacterium shimoidei, Mycobacterium branderi, Mycobacterium xenopi, Mycobacterium timonense, Mycobacterium parascrofulaceum, Mycobacterium heidelbergense, Mycobacterium rhizamassiliense, Mycobacterium numidiamassiliense, Mycobacterium gordonae, Mycobacterium manitobense, Mycobacterium lehmannii, Mycobacterium syngnathidarum, Mycobacterium arosiense, Mycobacterium colombiense, Mycobacterium terramassiliense, Mycobacterium decipiens, Mycobacterium talmoniae, Mycobacterium botniense, Mycobacterium genavense, Mycobacterium yunnanensis, Mycobacterium angelicum, Mycobacterium neglectum, Nocardia cerradoensis, Nocardia nova, Nocardia exalbida, Nocardia transvalensis, Mycobacterium simulans, and/or Mycobacterium riyadhense.

Typically, clinical polymicrobial infections include a median of about 6 species or fewer, and seldom have more than 10 species. Clinical polymicrobial infections internally typically have fewer than 6 species (e.g., 3 organism groups). The present systems and methods may detect identical bases from multiple species (e.g., from 3 organism groups or fewer) at the same sequencing position to identify the multiple species. Rather than isolating the different species in a sample from a polymicrobial infection prior to sequencing, the techniques described herein may conduct sequencing of the mixed sample, producing a chromatogram with information about the different species. By identifying bases from multiple species at the same sequencing position, the techniques described herein may detect and identify the multiple species in the sample. By reducing the number of sequencing runs needed to identify different species in a polymicrobial infection, the techniques described herein reduce the amount of laboratory and computational resources. These techniques also improve accuracy and efficiency of detection and identification of species in a polymicrobial samples. These and other improvements are detailed herein.

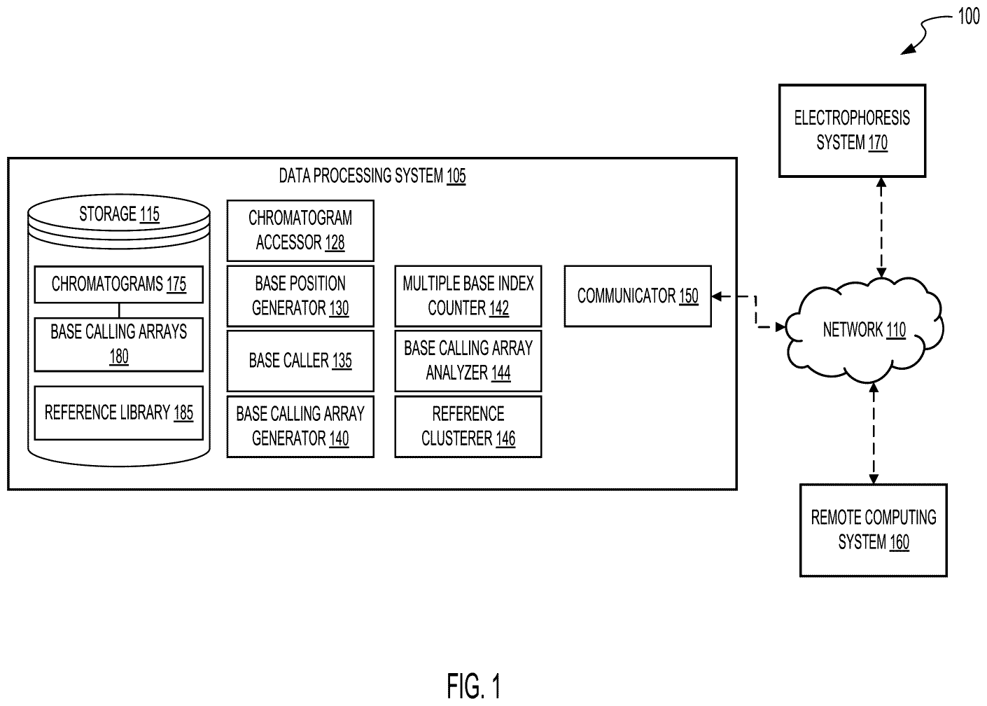

Referring to FIG. 1, illustrated is a block diagram of an example system 100 for identifying multiple species in chromatograms of mixed samples, in accordance with one or more implementations. The system 100 can include at least one data processing system 105, at least one network 110, and at least one remote computing system 160. The data processing system 105 can include a base position generator 130, a base caller 135, a base calling array generator 140, and storage 115. In some implementations, the data processing system 105 may additionally include a multiple base index counter 142, base calling array analyzer 144, and a reference clusterer 146. The storage 115 can include chromatograms 175, base calling arrays 180, and reference library 185. Although shown here as internal to the data processing system 105, the storage 115 can be external to the data processing system 105, for example, as a part of a local storage repository.

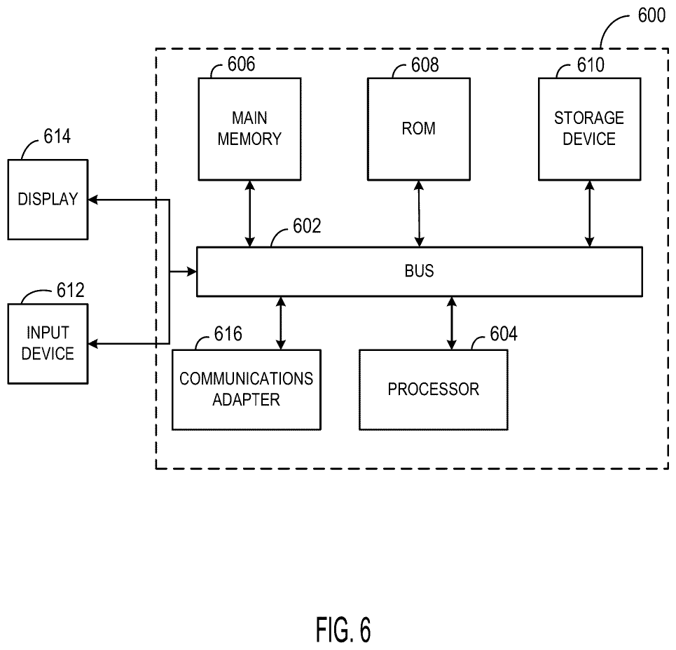

Each of the components (e.g., the data processing system 105, the network 110, the remote computing system 160, the base position generator 130, the base caller 135, the base calling array generator 140, the electrophoresis system 170, the communicator 150, the storage 115, etc.) of the system 100 can be implemented using the hardware components or a combination of software with the hardware components of a computing system, such as the computing system 600 described in connection with FIG. 6, or any other computing system described herein. Each of the components of the data processing system 105 can perform the functionalities detailed herein.

The data processing system 105 can include at least one processor and a memory (e.g., a processing circuit). The memory can store processor-executable instructions that, when executed by a processor, cause the processor to perform one or more of the operations described herein. The processor may include a microprocessor, an application-specific integrated circuit (ASIC), a field-programmable gate array (FPGA), a graphics processing unit (GPU), etc., or combinations thereof. The memory (which may be or include the storage 115) may include, but is not limited to, electronic, optical, magnetic, or any other storage or transmission device capable of providing the processor with program instructions. The memory may further include a floppy disk, CD-ROM, DVD, magnetic disk, memory chip, ASIC, FPGA, read-only memory (ROM), random-access memory (RAM), electrically erasable programmable ROM (EEPROM), erasable programmable ROM (EPROM), flash memory, optical media, or any other suitable memory from which the processor can read instructions. The instructions may include code from any suitable computer programming language. The data processing system 105 can include one or more computing devices or servers that can perform various functions as described herein. The data processing system 105 can include any or all of the components and perform any or all of the functions of the computer system 600 described in connection with FIG. 6.

In some implementations, the data processing system 105 may communicate with the remote computing system 160, for example, to provide information (e.g., chromatograms 175 and/or base calling arrays 180 in the storage 115). In one example, the data processing system 105 can be or can include an application server or webserver, which may include software modules to access or transmit chromatograms 175 and/or base calling arrays 180 stored by the data processing system 105 (e.g., in the storage 115). For example, the data processing system 105 may include a webserver allowing the remote computing system 160 to request one or more chromatograms, base calling arrays, or other genetic data corresponding to one or more samples. In response, the data processing system 105 can transmit the chromatograms, base calling arrays, or other genetic data to the remote computing system 160. In some implementations, the data processing system 105 can transmit data to the remote computing system 160 in response to user input by an operator of the data processing system 105.

The network 110 can include computer networks such as the Internet, local, wide, or other area networks, intranets, and satellite networks, other computer networks such as voice or data mobile phone communication networks, or combinations thereof. The data processing system 105 of the system 100 can communicate via the network 110 with one or more computing devices, such as the remote computing system 160. The network 110 may be any form of computer network that can relay information between the data processing system 105 and the remote computing system 160. In some implementations, the network 110 may include the Internet and/or other types of data networks, such as a local area network (LAN), a wide area network (WAN), a cellular network, a satellite network, or other types of data networks. The network 110 may also include any number of computing devices (e.g., computers, servers, routers, network switches, etc.) that are configured to receive or transmit data within the network 110.

The network 110 may further include any number of hardwired or wireless connections. Any or all of the computing devices described herein (e.g., the data processing system 105, the remote computing system 160, the computer system 600, etc.) may communicate wirelessly (e.g., via Wi-Fi, cellular communication, radio, etc.) with a transceiver that is hardwired (e.g., via a fiber optic cable, a CAT5 cable, etc.) to other computing devices in the network 110. Any or all of the computing devices described herein (e.g., the data processing system 105, the remote computing system 160, the computer system 600, etc.) may also communicate wirelessly with the computing devices of the network 110 via a proxy device (e.g., a router, network switch, or gateway).

The remote computing system 160 can include at least one processor and a memory (e.g., a processing circuit). The memory can store processor-executable instructions that, when executed by the processor, cause the processor to perform one or more of the operations described herein. The processor can include a microprocessor, an ASIC, an FPGA, a GPU, etc., or combinations thereof. The memory can include, but is not limited to, electronic, optical, magnetic, or any other storage or transmission device capable of providing the processor with program instructions. The memory can further include a floppy disk, CD-ROM, DVD, magnetic disk, memory chip, ASIC, FPGA, ROM, RAM, EEPROM, EPROM, flash memory, optical media, or any other suitable memory from which the processor can read instructions. The instructions can include code from any suitable computer programming language. The remote computing system 160 can include one or more computing devices, servers, personal computing devices, or data repositories. The remote computing system 160 can include any or all of the components and perform any or all of the functions of the computer system 600 described in connection with FIG. 6.

The storage 115 can be a computer-readable memory that can store or maintain any of the information described herein. The storage 115 can store or maintain one or more data structures, which may contain, index, or otherwise store each of the values, pluralities, sets, variables, vectors, numbers, or thresholds described herein. The storage 115 can be accessed using one or more memory addresses, index values, or identifiers of any item, structure, or region maintained in the storage 115. In implementations where the storage 115 is external to the data processing system 105, the storage 115 can be accessed by the components of the data processing system 105 via the network 110 or via a local communications interface. The storage 115 can be distributed across many different computer systems or storage elements. The data processing system 105 can store, in one or more regions of the memory of the data processing system 105, or in the storage 115, the results of any or all computations, determinations, selections, identifications, generations, constructions, or calculations in one or more data structures indexed or identified with appropriate values.

Any or all values stored in the storage 115 may be accessed by the components of the data processing system 105 to perform any of the functionalities or functions described herein. In some implementations, the data processing system 105 may utilize authentication information (e.g., username, password, email, etc.) to show that an operator of the data processing system 105 is authorized to access requested information in the storage 115. The storage 115 may include permission settings that indicate which users, devices, or profiles are authorized to access certain information stored in the storage 115.

The storage 115 can store one or more chromatograms 175, for example, in one or more data structures or files. The chromatograms 175 can be generated by the electrophoresis system 170 and transferred from the electrophoresis system 170 to the storage 115 via the network 110. The electrophoresis system 170 may have the capability of conducting DNA sequencing to produce chromatograms 175 representing DNA sequences of one or more organisms. For example, the electrophoresis system 170 may be capable of conducting Sanger sequencing to produce chromatograms 175. Sanger sequencing is a method of DNA sequencing based on the random incorporation of chain-terminating dideoxynucleotides by DNA polymerase during in vitro DNA replication. Although shown here as external to the data processing system 105, the electrophoresis system 170 can be internal to the data processing system 105, for example, as shared processors and/or memory.

In some implementations, the data processing system 105 may communicate with the electrophoresis system 170, for example, to receive or provide information (e.g., chromatograms 175 and/or base calling arrays 180). In one example, the data processing system 105 can be or can include an application server or webserver, which may include software modules to access or transmit data stored by the data processing system 105 (e.g., in the storage 115). For example, the data processing system 105 may include a webserver allowing the data processing system 105 to request one or more chromatograms, base calling arrays, or other genetic data corresponding to one or more samples. In response, the electropherogram 170 can transmit the chromatograms 175, base calling arrays 180, or other genetic data to the data processing system 105. In some implementations, the electrophoresis system 170 can transmit data to the data processing system 105 in response to user input by an operator of the data processing system 105.

The electrophoresis system 170 may include at least one processor and a memory (e.g., a processing circuit). The memory can store processor-executable instructions that, when executed by the processor, cause the processor to perform one or more of the operations described herein. The memory can also store chromatograms 175 collected during sequencing runs. The processor can include a microprocessor, an ASIC, an FPGA, a GPU, etc., or combinations thereof. The memory can include, but is not limited to, electronic, optical, magnetic, or any other storage or transmission device capable of providing the processor with program instructions. The memory can further include a floppy disk, CD-ROM, DVD, magnetic disk, memory chip, ASIC, FPGA, ROM, RAM, EEPROM, EPROM, flash memory, optical media, or any other suitable memory from which the processor can read instructions.

The chromatograms 175 may include 1 to 4 signals (e.g., 4 signals), each signal representing a different DNA base (e.g. A, T, G, or C). The chromatograms 175 may include a time axis and an intensity axis, and the signals may represent the results from a sequencing run. Peaks in the signals may represent migration of labeled sequencing products via capillary electrophoresis. For example, fluorescence may be detected at the end of the capillary, and signal intensity for each signal, corresponding to a DNA base, may be plotted on the intensity y-axis relative to migration time on the x-axis. The chromatograms 175 may be used to determine partial or entire nucleotide sequences to identify one or more species in metagenomic samples, as described in more detail below.

Nucleotide sequences determined from chromatograms 175 may be represented in base calling arrays 180. The storage 115 can store one or more base calling arrays 180, for example, in one or more data structures or files. Each base calling array may include one or more sequential integer indexes. Each index may include one or more DNA bases associated with that index, with multiple DNA bases resulting from multiple species in the sequencing sample. DNA bases in a given index may be determined from a chromatogram, where each index may correspond to a window (i.e., a portion) of a chromatogram along the time axis. The storage 115 may store multiple base calling arrays that each correspond to a respective chromatogram and each chromatogram may correspond to a respective DNA sequencing run, as described herein.

For example, each base calling array 180 can be generated from a corresponding chromatogram 175. As described herein, chromatograms 175 can include genetic information from a metagenomic sample. The chromatogram, which corresponds to a DNA sequence from the sample, can include a number of sequential DNA bases. The base calling array 180 corresponding to respective chromatogram can include indexes corresponding to overlapping windows in the chromatogram (of a predetermined length, e.g., the predetermined length of an average anchor peak), with DNA bases in the indexes extracted from the chromatogram. For example, the base calling array 180 can be generated by iterating through a chromatogram window-by-window, and adding all DNA bases in a given window to the corresponding index, and shifting one window at a time. The base calling array 180 can be utilized by the components of the data processing system 105 to identify species present in the metagenomic sample.

The reference library 185 may include one or more DNA sequences representing different species or organisms relevant to the type of sample to be identified. Sequences in the reference library 185 may be compared to DNA sequences in the base calling arrays 180 to identify species in the base calling arrays 180.

Referring now to the operations of the components of the data processing system 105, the chromatogram accessor 128 can access one or more chromatograms 170 that represent DNA sequencing data. The chromatogram can be, for example, sequencing data from a pathogenic sample. As described herein, each chromatogram 170 can correspond to the full or partial DNA sequence of one or more species in the sample. The chromatogram accessor 128 can access each chromatogram 170 iteratively, for example, to generate a base calling array using the contents of the chromatogram 170, as described herein. For example, the chromatogram accessor 128 can be utilized to access a chromatogram 170 for a respective sample per iteration, until chromatograms 170 have been accessed and base calling arrays 180 have been generated for each sample. Chromatograms 170 may be extracted from files. The file can be any suitable file, including a CSV format file, an XML format file, a TXT format file, an GEXF format file, a GML format file, or a GDF format file, among others. When accessing the file corresponding to the sequencing run, the chromatogram accessor 128 can parse header information of the file to sequencing run data.

The base position generator 130 generates one or more base positions along the time axis of the chromatogram corresponding to the chromatogram data. To do so, the base position generator 130 may generate a base position on the time axis for every peak or inflection point (i.e., (where the signal curvature changes from concave down to up or vice versa) in respective chromatogram signals. In some implementations, the base position generator 130 may generate multiple base positions at wide peaks, where wide peaks are defined as having a full width half max (FWHM) greater than a predetermined width. The predetermined width may be one or more of a fixed numerical value, a fixed value relative to an average peak FWHM, or a fixed value relative to an anchor peak FWHM (or an average anchor peak FWHM).

In some implementations, the predetermined width may be a fixed value relative to an anchor peak FWHM. The predetermined width relative to the anchor peak may be 1.1X to 2.5X (e.g., 1.1X, 1.2X, 1.3X, 1.4X, 1.5X, 1.6X, 1.7X, 1.8X, 1.9X, 2X, 2.1X, 2.2X, 2.3X, 2.4X, or 2.5X), where X is a FWHM of the anchor peak. For example, the predetermined width may be about 1.5X.

For each wide peak, the base position generator 130 may generate multiple base positions. The position and number of base positions assigned to each wide peak varies depending on the shape of the peak and the width of the peak. Two base positions may be generated if the wide peak width is within a threshold width range, and three base positions may be generated if the wide peak width is greater than the threshold width range. For example, if the wide peak width is 1.5X to 2X, two base positions may be generated; and if the wide peak width is greater than 2X, then three base positions may be generated. The base position generator 130 identifies whether the wide peak skews left, right, or is centered to determine where to place the base positions corresponding to the wide peak. Peak skew is identified based on difference in distance from the top of the peak to the bottom of each side of peak. For wide peaks generating two base positions that skew left, one base position may be generated at (½)X to 1X (e.g., ( 4/6)X, (⅚)X, or 1X) to the left of the wide peak's apex and a second base position may be generated at ( 1/12)X to ( 2/6)X (e.g., (⅙)X or ( 3/12)X) to the right of the wide peak's apex. For wide peaks generating two base positions that skew right, a base position may be at (½)X to 1X (e.g., ( 4/6)X, (⅚)X, or 1X) to the right of the wide peak's apex and a second base position may be generated at ( 1/12)X to ( 2/6)X (e.g., (⅙)X or ( 3/12)X) to the left of the wide peak's apex. For wide peaks generating two base positions that are substantially centered, base positions may be generated equidistant from the wide peak's apex. For example, base positions for a centered wide peak may be generated at ( 2/6)X to (½)X) (e.g., ( 5/12)X)) to the left and right of the wide peak's apex.

For example, if an anchor peak has a FWHM of 12 time units (e.g., minutes), then the base position generator 130 may generate base positions in the following ways. For wide peaks skewing left, base positions may be generated 10 time units to the left of the apex and 2 time units to the right of the apex. For wide peaks skewing right, base positions may be generated 10 time units to the right of the apex and 2 time units to the left of the apex. For centered wide peaks, base positions may be generated 5 time units to the left and right of the apex.

The base caller 135 calls a base at each base position generated by the base position generator 130. The base caller 135 calls bases according to the signals in the chromatogram, where respective signals represent a different DNA base. After the base caller 135 calls the bases, bases are present in the positions where they were detected along the time axis of the chromatogram.

Once the base caller 135 calls the bases along the time axis of the chromatogram, the base calling array generator 140 generates a base calling array representing the bases to gather close bases to the same position. The base calling array includes one or more indexes. Each index corresponds to a window along (i.e., a portion of) the time axis such that called bases within the window are added to the corresponding index. In this way, each index acts as a bucket or a slot into which bases within the corresponding window are added.

The index window may have a predetermined width corresponding to a fixed number of time units. The predetermined width may be such that a window partially overlaps with adjacent windows. With overlapping windows, a base in the middle of two indexes may be placed twice, that is, once in each neighboring index. For example, if an anchor peak has a FWHM of 12 time units (e.g., minutes), then the window may be 12 time units to 24 time units, including 13, 14, 15, 16, 17, 18. 19, 20, 21, 22, 23, or 24 time units. In some implementations, the window is 18 time units.

In any implementation, the base calling array generator 140 may identify one or more anchor peaks in the chromatogram. One or more anchor peaks may be used to orient the index windows to the time axis. To do so, the base calling array generator 140 may assign one or more anchor peaks as midpoints of the respective index windows in the base calling array. For example, for each index window, the base calling array generator 140 may identify an anchor peak if the anchor peak is present in the index window. If the anchor peak is present, the base calling array generator 140 may adjust the index window so that the midpoint of the anchor peak is the midpoint of the window. This adjustment may compensate for artifacts or errors in sequencing data, including unevenly spaced bases (e.g., due to different organisms having different separation behavior) and loss of resolution.

The base calling array generator 140 may generate the base calling array by iteratively adding bases corresponding to an index window to the respective index until all called bases are added to the base calling array. If the base calling array generator 140 adjusts an index window to match an anchor peak, the base calling array generator 140 may shift all subsequent index windows (i.e., to the right of the window that is adjusted) in the same way. If the base calling array generator 140 does not detect an anchor peak in the index window, then the base calling array generator 140 does not change the position of the index window.

In some implementations, the base calling array generator 140 detects the first anchor peak as the first anchor peak along the time axis, and uses the first anchor peak as the midpoint of the first index window for generating the base calling array.

Base calling arrays generated by the base calling array generator 140 may in a text-based format (or a compressed format thereof using a suitable compression algorithm). For example, the file format may be a FAST fil format, a CSV format file, an XML format file, or a TXT format file, among others.

Base calling arrays generated by the base calling array generator 140 may be used to identify one or multiple species present in the sequenced sample by, for example, comparing the sequences in the base calling array to a reference library. Base calling arrays may be stored in storage 115. Base calling arrays may be accessed by the electrophoresis system 170 and/or the remote computing system 160 via the network 110. In some implementations, organism identification occurs on the remote computing system 160 or the electrophoresis system 170. For example, the communicator 150 may transmit a data package that includes a base calling array to the remote computing system 160 via the network 110. The data package may include additional metadata that includes information about the data package or the base calling array (e.g., information about sample run). In some implementations, the communicator 150 can compress the data package using a suitable compression algorithm prior to transmission, to reduce the utilization of network resources. The communicator 150 can transmit the data package to the remote computing system 160 in response to a request, or in response to an operator of the data processing system 105 providing user input. In some implementations, the data processing system 105 includes a sequence identifier that can match the sequences in the base calling array to the reference library.

The multiple base index counter 142 counts the number of indexes in base calling arrays with three or more bases and/or with four or more bases. A base calling array with a number of indexes with three or more bases and/or four or more bases greater than a predetermined threshold may indicate that the base calling array data is ambiguous and/or low-quality. The multiple base index counter 142 counts the number of indexes with three bases and the number of indexes with four bases. The multiple base index counter 142 may compare the counted number of indexes with three and/or four bases to predetermined thresholds. If the multiple base index counter 142 determines that the number of indexes with multiple indexes exceeds a threshold, the multiple base index counter 142 may send a notification to a client device (e.g., the remote computing system 160, or the electrophoresis system 170).

For example, the predetermined threshold for the number of indexes with three or more bases is 30%, 40%, 50%, 60%, 70%, or 80% of the indexes. In some implementations, different predetermined thresholds may be used for the number of indexes with three bases and the number of indexes with four bases. For example, a predetermined threshold for the number of indexes with three bases may be 30%, 40%, 50%, 60%, 70%, or 80% of the indexes. For example, a predetermined threshold for the number of indexes with four bases may be 10%, 20%, 30%, 40%, or 50% of the indexes. In some implementations, the data processing system uses more than one predetermined threshold, where exceeding the lower threshold results in sending a notification to the client device and exceeding the higher threshold results in sending a notification to the client device and stopping data processing. For example, a lower predetermined threshold number of indexes with three bases may be 40%, 50%, or 60%; and the higher predetermined threshold number of indexes with three bases may be 60%, 70%, or 80%. For example, a lower predetermined threshold number of indexes with four bases may be 10%, 20%, or 30%; and the higher predetermined threshold number of indexes with four bases may be 20%, 30%, or 40%. As an example, for the threshold number of indexes with three bases, the lower value may be 50% and the higher value may be 70%.

The base calling array analyzer 144 compares DNA sequences (e.g., those in base calling arrays generated by the base calling array generator 140) to the reference library 185 to identify the species in the sample. The base calling array analyzer 144 may iteratively compare reference sequences from the reference library 185 to the base calling array sequence to determine a percent match, and rank reference species according to their percent match. In some implementations, references with a percent match below a threshold value are discarded from the results.

The reference clusterer 146 clusters reference sequences above a percent match threshold identified by the base calling array analyzer 144 in order to improve match results. Reference sequences may be grouped according to Levenshtein distance. The Levenshtein distance between two sequences is a measure of the number of single-base edits (insertions, deletions, or substitutions) to change one sequence into the other sequence. The Levenshtein distance between each reference sequence may be determined, and reference sequences with distances less than a threshold value may be clustered together into groups of similar sequences. For example, the threshold value may be 3%-8%, 4%-6%, 1%, 2%, 3%, 4%, 5%, 6%, 7%, 8%, 9%, or 10%. If the number of groups exceeds a threshold number, then the reference clusterer 146 may send a client device notification indicating the possibility of poor-quality data. The reference clusterer 146 may discard the data if there are too many groups. Otherwise, the reference clusterer 146 may report the groups to the client device.

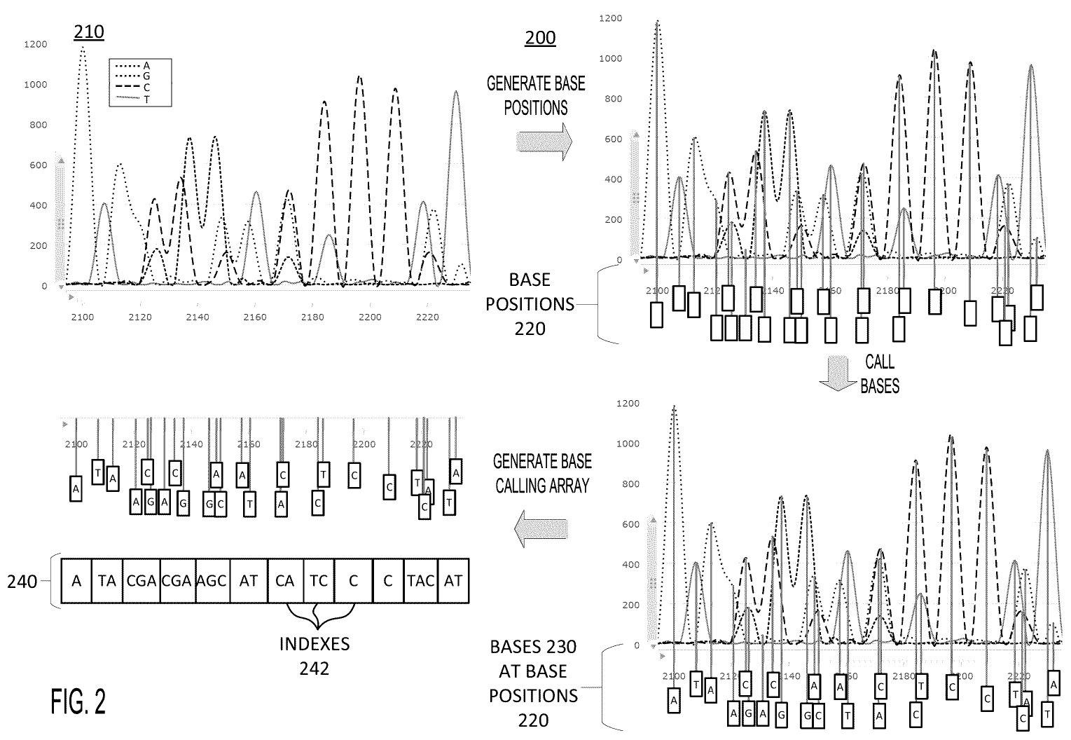

Referring to FIG. 2, illustrated is an example dataflow diagram 200 of a process for identifying multiple species in chromatograms of mixed samples, in accordance with one or more implementations. The process shown in the diagram 200 can be performed, for example, by the data processing system 105 described in connection with FIG. 1. As shown, a chromatogram 210 (e.g., having 4 signals representing different DNA bases) can be used to generate a base calling array 240 that includes multiple indexes 242 to gather close bases together and compensate for sampling errors or artifacts. The chromatogram 210 may include one or multiple signals, with each signal representing a different DNA base. The chromatogram 210 may have a time axis (e.g., corresponding to electrophoresis migration time) and an intensity axis (e.g., corresponding to a fluorescence intensity from fluorescently labelled DNA fragments).

To do so, base positions 220 are generated at every peak and inflection point in the signals in the chromatogram 210. In some implementations, multiple base positions are generated at wide peaks, as described in more detail above with respect to FIG. 1. Base positions 220 are generated along the time axis of the chromatogram 210.

After base positions 220 are generated, DNA bases 230 can be called at base positions 220, with a DNA base called for every base position. Bases can be called according to the respective signal in the chromatogram 210. After the bases are called, bases may be present at their respective positions along the time axis of the chromatogram 210.

Once bases are called, a base calling array 240 (e.g., base calling array 180 of FIG. 1) can be generated to gather close bases together and compensate for sampling errors or artifacts in mixed samples. The base calling array 240 includes multiple indexes 242, with each index corresponding to a window of the chromatogram 210, such that called bases within the window are added to the corresponding index. The size and position of respective index windows may be adjusted as described above with respect to FIG. 1.

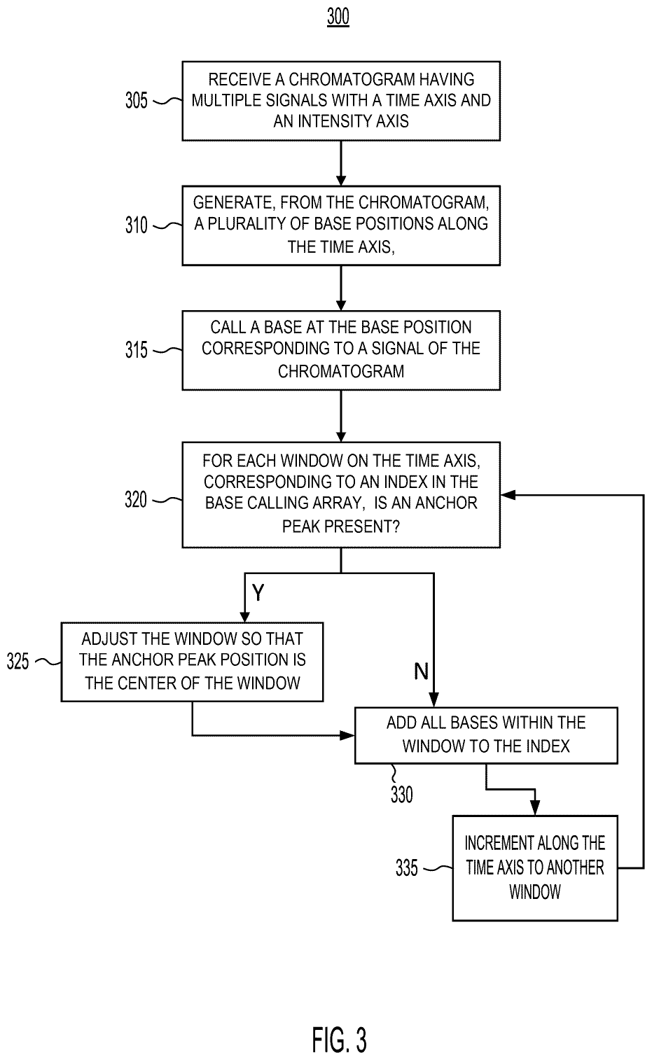

FIG. 3 illustrates a flow chart of an example method 300 for identifying multiple species in chromatograms of mixed samples, in accordance with one or more implementations. The method 300 can be performed, for example, by a data processing system (e.g., the data processing system 105), or any computing devices described herein (e.g., the computer system 600 of FIG. 6). It should be understood that the method 300 shown in FIG. 3 is an example, and that additional steps may be performed, steps may be omitted, or steps may be performed in a different order than shown, to achieve desired results.

At act 305, the method 300 includes accessing a chromatogram representing a sequencing run (e.g., a Sanger sequencing run). The chromatogram may include one or multiple signals, with each signal representing a different DNA base. The chromatogram may have a time axis (e.g., corresponding to electrophoresis migration time) and an intensity axis (e.g., corresponding to a fluorescence intensity from fluorescently labelled DNA fragments). The data processing system can access each chromatogram iteratively, for example, to generate respective base calling arrays, as described herein. For example, the data processing system can be utilized to access chromatogram for a respective sample per iteration, until all chromatograms have been accessed and respective base calling arrays have been generated.

In some implementations, the data processing system can receive the chromatogram(s) from an electrophoresis system (e.g., electrophoresis system 170) via a network. Each chromatogram can correspond to a DNA sequence of one or more species (e.g., pathogenic species) in a sequencing sample. The files used to represent the chromatograms as described herein can be formed in a graphical format or text-based format (or a compressed format thereof using a suitable compression algorithm). For example, the file format can be any suitable file, including a CSV format file, an XML format file, a TXT format file, an GEXF format file, a GML format file, or a GDF format file, among others. When accessing the file corresponding to the sequencing run, the header information from the file may be parsed to sequencing run data.

The received chromatogram(s) may be stored in a storage (e.g., storage 115) and accessed by a data processing system (e.g., data processing 105) to perform any of the functionalities or functions described herein.

At act 310, the method 300 includes generating a plurality of base positions along the time axis of the chromatogram. As described herein, a base position may be generated on the time axis for every peak or inflection point (i.e., (where the signal curvature changes from concave down to up or vice versa) in respective chromatogram signals. In some implementations, multiple base positions are generated at wide peaks, where wide peaks are defined as having a full width half max (FWHM) greater than a predetermined width, as described herein.

At act 315, the method 300 includes calling a base at every base position corresponding to the signal of the chromatogram, where respective signals represent a different DNA base (e.g., A, C, G, or T). After the bases are called, bases are present in the positions where they were detected along the time axis of the chromatogram.

After calling bases at act 315, a base calling array (e.g., base calling array 180 of FIG. 1 or the base calling array 240 of FIG. 2) may be generated that gathers close bases to the same position. The base calling array may be generated using time windows that correspond to respective indexes in the base calling array. The windows may be predetermined to have a width corresponding to a fixed number of time units. The width may be such that neighboring windows partially overlap with another. With overlapping windows, a base in the middle of two indexes may be placed twice, that is, once in each neighboring index. For example, if an anchor peak has a FWHM of 12 time units (e.g., minutes), then the window may be 12 time units to 24 time units, including 13, 14, 15, 16, 17, 18. 19, 20, 21, 22, 23, or 24 time units. In some implementations, the window is 18 time units.

At act 320, the method 300 includes, for each window on the time axis corresponding an index in the base calling array, determining the presence of an anchor peak. The data processing system can use anchor peaks to adjust the position of the windows to compensate for artifacts or errors in the sequencing data, such as unevenly spaced bases (e.g., due to different organisms having different separation behavior) and loss of resolution. If an anchor peak is present in the window, at act 325, the method 300 includes adjusting the window so that the anchor peak position is the center of the window. This window adjustment may shift all subsequent windows (i.e., windows to the right of the window that is adjusted) in the same way.

Whether or not an anchor peak is present in the window, the method 300 includes, at act 330, adding all called bases within the window to the corresponding index. In this way, close bases (e.g., from multiple species) are gathered together into the same index in the base calling array.

At act 335, the method 300 includes incrementing along the time axis of the chromatogram to the next overlapping window and repeating acts 320-335 to gathers close bases into indexes. This process can be repeated to generate the base calling array corresponding to the chromatogram.

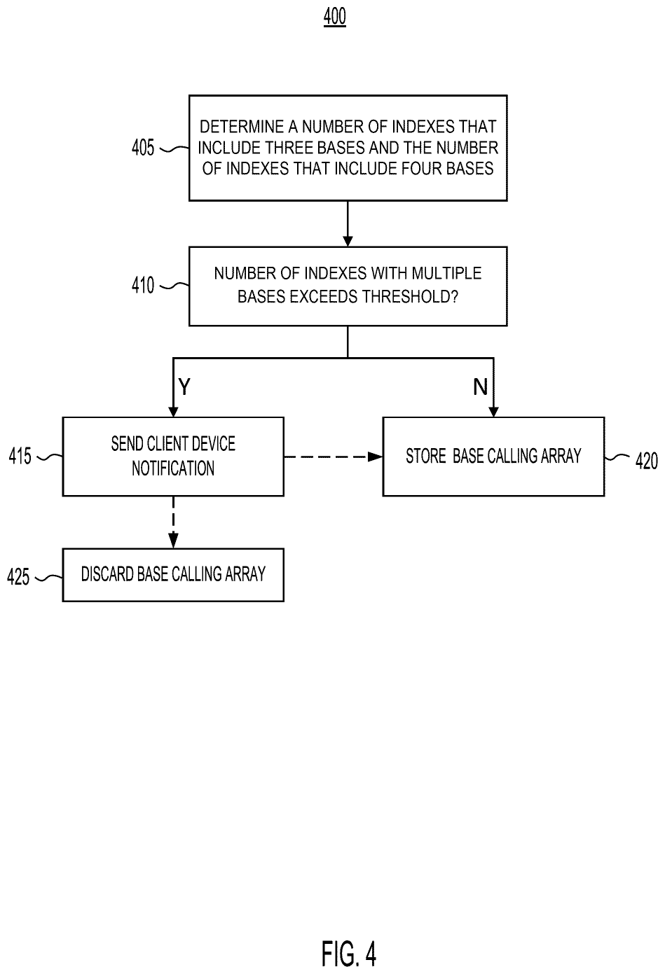

FIG. 4 illustrates a flow chart of an example method for notifying a client device if a base calling array includes too many indexes with multiple bases, in accordance with one or more implementations. If more than a predetermined threshold number of indexes include multiple bases (e.g., three or more bases in an index), then the data processing system may alert the client device that the data may be inaccurate. Having a greater number of indexes with three or more bases may indicate ambiguous and/or low-quality data. The method 400 may decrease computational resources used to analyze a base calling array by discarding bad data. The method 400 may be performed by the data processing system, for example, after the base calling array is generated but before performing an analysis of the base calling array sequence(s) to identify species. The method 400 can be performed, for example, by a data processing system (e.g., the data processing system 105), or any computing devices described herein (e.g., the computer system 600 of FIG. 6). It should be understood that the method 400 shown in FIG. 4 is an example, and that additional steps may be performed, steps may be omitted, or steps may be performed in a different order than shown, to achieve desired results.

At act 405, the method 400 includes determining a number of indexes in a base calling array (e.g., base calling array 180 of FIG. 1) that include three bases and the number of indexes that include four bases.

At act 410, the method 400 includes determining if the number of indexes with three bases and/or the number of indexes with four bases exceeds a predetermined threshold. In some implementations, the predetermined threshold may be based on the cumulative number of indexes with three or four bases.

For example, the predetermined threshold may be the number of indexes with more than three bases that is 30%, 40%, 50%, 60%, 70%, or 80% of the indexes. In some implementations, different predetermined thresholds may be used for the number of indexes with three bases and the number of indexes with four bases. For example, a predetermined threshold for the number of indexes with three bases may be 30%, 40%, 50%, 60%, 70%, or 80% of the indexes. For example, a predetermined threshold for the number of indexes with four bases may be 10%, 20%, 30%, 40%, or 50% of the indexes. In some implementations, the data processing system uses more than one predetermined threshold, where exceeding the lower threshold results in sending a notification to the client device and exceeding the higher threshold results in sending a notification to the client device and stopping data processing. For example, a lower predetermined threshold number of indexes with three bases may be 40%, 50%, or 60%; and the higher predetermined threshold number of indexes with three bases may be 60%, 70%, or 80%. For example, a lower predetermined threshold number of indexes with four bases may be 10%, 20%, or 30%; and the higher predetermined threshold number of indexes with four bases may be 20%, 30%, or 40%. As an example, for the threshold number of indexes with three bases, the lower value may be 50% and the higher value may be 70%. As an example, for the threshold number of indexes with four bases, the lower value may be 20% and the higher value may be 30%.

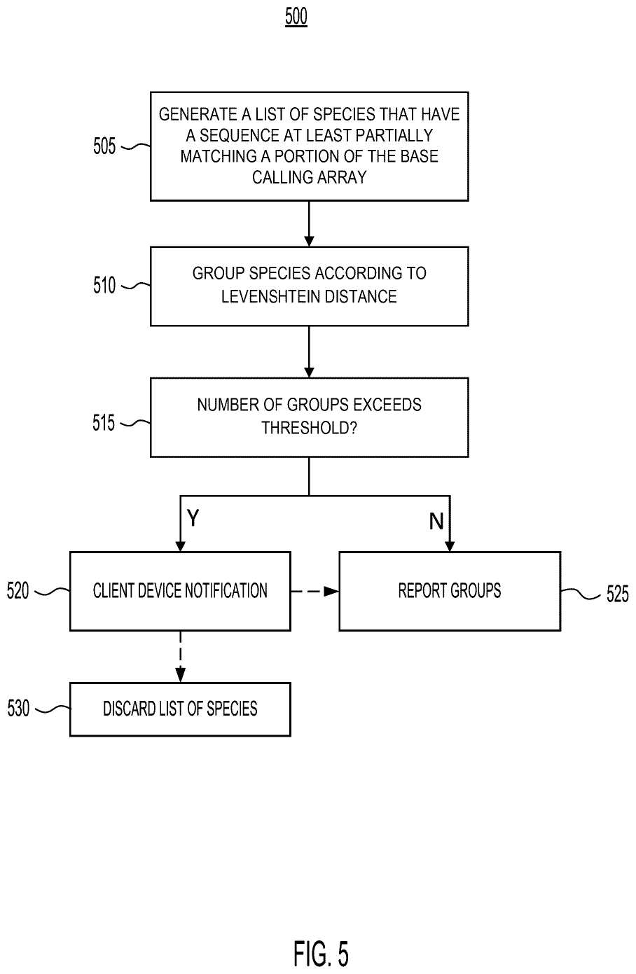

At act 415 of the method 400, if the predetermined threshold number of indexes with three or more bases is exceeded, the data processing system sends a client device notification alerting a user of the possibility of ambiguous and/or low-quality data. In some implementations, after sending the client device notification, the data processing system may discard the base calling array at act 425 of the method 400. For example, the data processing system may discard the base calling array if the number of indexes with three or more bases exceeds the higher threshold described above. In some implementations, after sending the client device notification, the data processing system may, at act 420 of the method 400, store the base calling array in storage (e.g., storage 115 of FIG. 1) or analyze the data to identify species matching the DNA sequence(s) in the base calling array (e.g., using the method 500 of FIG. 5).

If, at act 410, the data processing system determines that the number of indexes with three or more bases does not exceed the threshold(s), the data processing system may, at act 420 of the method 400, store the base calling array in storage (e.g., storage 115 of FIG. 1) or analyze the data to identify species matching the DNA sequence(s) in the base calling array (e.g., using the method 500 of FIG. 5).

FIG. 5 illustrates a flow chart of an example method 500 for analyzing base calling arrays, in accordance with one or more implementations. The analysis method 500 includes reference clustering to improve sequence matching and decrease computational resources. The method 500 clusters reference DNA sequences to improve matching of the sequences in the base calling arrays to the reference DNA sequences. The method 500 may be performed by the data processing system, for example, after the base calling array is generated and/or after the method 400 has been performed. The method 500 can be performed, for example, by a data processing system (e.g., the data processing system 105), or any computing devices described herein (e.g., the computer system 600 of FIG. 6). It should be understood that the method 500 shown in FIG. 5 is an example, and that additional steps may be performed, steps may be omitted, or steps may be performed in a different order than shown, to achieve desired results.

At act 505 of method 500, the data processing system generates a list of species that have a sequence at least partially matching a portion of the base calling array. For example, the data processing system may compare the base calling array sequences to one or more libraries of reference DNA sequences (e.g., reference library 185) to determine how closely the sequences match according to a percent match. The data processing system may rank the reference sequences according to percent match. In some implementations, the data processing system may discard reference sequences from the results if they fall below a threshold percent match.

At act 510 of method 500, the data processing system may determine Levenshtein distances between each reference sequence in the ranked reference sequences. From the Levenshtein distances, similarities may be determined as:

$$similarity = 100 - \frac{Levenshtein distance between reference I and N}{no . of bases in the shorter sequence of references I and N} \times 100$$

At act 510, the data processing system may use Levenshtein distances and/or similarity values to group species. Reference sequences with distances less than a threshold Levenshtein distance and/or greater than a threshold similarity may be clustered together into groups of similar sequences. For example, the threshold Levenshtein distance may be 3%, 4%, 5%, 6%, 7%, 8%, 9%, or 10%. For example, the threshold similarity may be 90%, 91%, 92%, 93%, 94%, 95%, 96%, 97%, 98%, or 99%. In some implementations, the data processing system may iterate this process to determine Levenshtein distances and/or similarities within one or more groups to separate groups into sub-groups. The threshold Levenshtein distance and/or similarity may be different in different iterations. For example, the threshold similarity for the first iteration may be 97% and the threshold similarity for the second iteration may be 99%.

At act 515 of the method 500, the data processing system may determine if the number of groups clustered according to Levenshtein distance at act 510 exceeds a predetermined threshold. The predetermined threshold may be 2, 3, 4, 5, 6, 7, 8, 9, or 10.

At act 520 of the method 500, if the number of groups exceeds the predetermined threshold, the data processing system sends a client device notification alerting a user of the possibility of ambiguous and/or low-quality data. In some implementations, after sending the client device notification, the data processing system may discard the generated list of species at act 530. In some implementations, after sending the client device notification, the data processing system may, at act 525 report the groups to a client device. If, at act 515, the data processing system determines that the number of groups exceeds the threshold, the data processing system may, at act 525 of the method, report the groups to the client device.

FIG. 6 is a component diagram of an example computing system suitable for use in the various implementations described herein, according to an example implementation. For example, the computing system 600 may implement the data processing system 105 or the remote computing system 160 of FIG. 1, or various other example systems and devices described in the present disclosure.

The computing system 600 includes a bus 602 or other communication component for communicating information and a processor 604 coupled to the bus 602 for processing information. The computing system 600 also includes main memory 606, such as a RAM or other dynamic storage device, coupled to the bus 602 for storing information, and instructions to be executed by the processor 604. Main memory 606 can also be used for storing position information, temporary variables, or other intermediate information during execution of instructions by the processor 604. The computing system 600 may further include a ROM 608 or other static storage device coupled to the bus 602 for storing static information and instructions for the processor 604. A storage device 610, such as a solid-state device, magnetic disk, or optical disk, is coupled to the bus 602 for persistently storing information and instructions.

The computing system 600 may be coupled via the bus 602 to a display 614, such as a liquid crystal display, or active matrix display, for displaying information and notifications to a user. An input device 612, such as a keyboard including alphanumeric and other keys, may be coupled to the bus 602 for communicating information, and command selections to the processor 604. In another implementation, the input device 612 has a touch screen display. The input device 612 can include any type of biometric sensor, or a cursor control, such as a mouse, a trackball, or cursor direction keys, for communicating direction information and command selections to the processor 604 and for controlling cursor movement on the display 614.

In some implementations, the computing system 600 may include a communications adapter 616, such as a networking adapter. Communications adapter 616 may be coupled to bus 602 and may be configured to enable communications with a computing or communications network or other computing systems. In various illustrative implementations, any type of networking configuration may be achieved using communications adapter 616, such as wired (e.g., via Ethernet), wireless (e.g., via Wi-Fi, Bluetooth), satellite (e.g., via GPS) pre-configured, ad-hoc, LAN, WAN, and the like.

According to various implementations, the processes of the illustrative implementations that are described herein can be achieved by the computing system 600 in response to the processor 604 executing an implementation of instructions contained in main memory 606. Such instructions can be read into main memory 606 from another computer-readable medium, such as the storage device 610. Execution of the implementation of instructions contained in main memory 606 causes the computing system 600 to perform the illustrative processes described herein. One or more processors in a multi-processing implementation may also be employed to execute the instructions contained in main memory 606. In alternative implementations, hard-wired circuitry may be used in place of or in combination with software instructions to implement illustrative implementations. Thus, implementations are not limited to any specific combination of hardware circuitry and software.

The implementations described herein have been described with reference to drawings. The drawings illustrate certain details of specific implementations that implement the systems, methods, and programs described herein. However, describing the implementations with drawings should not be construed as imposing on the disclosure any limitations that may be present in the drawings.

It should be understood that no claim element herein is to be construed under the provisions of 35 U.S.C. § 112(f), unless the element is expressly recited using the phrase “means for.”

As used herein, the term “circuit” may include hardware structured to execute the functions described herein. In some implementations, each respective “circuit” may include machine-readable media for configuring the hardware to execute the functions described herein. The circuit may be embodied as one or more circuitry components including, but not limited to, processing circuitry, network interfaces, peripheral devices, input devices, output devices, sensors, etc. In some implementations, a circuit may take the form of one or more analog circuits, electronic circuits (e.g., integrated circuits (IC), discrete circuits, system on a chip (SOC) circuits), telecommunication circuits, hybrid circuits, and any other type of “circuit.” In this regard, the “circuit” may include any type of component for accomplishing or facilitating achievement of the operations described herein. For example, a circuit as described herein may include one or more transistors, logic gates (e.g., NAND, AND, NOR, OR, XOR, NOT, XNOR), resistors, multiplexers, registers, capacitors, inductors, diodes, wiring, and so on.

The “circuit” may also include one or more processors communicatively coupled to one or more memory or memory devices. In this regard, the one or more processors may execute instructions stored in the memory or may execute instructions otherwise accessible to the one or more processors. In some implementations, the one or more processors may be embodied in various ways. The one or more processors may be constructed in a manner sufficient to perform at least the operations described herein. In some implementations, the one or more processors may be shared by multiple circuits (e.g., circuit A and circuit B may comprise or otherwise share the same processor, which, in some example implementations, may execute instructions stored, or otherwise accessed, via different areas of memory). Alternatively or additionally, the one or more processors may be structured to perform or otherwise execute certain operations independent of one or more co-processors.

In other example implementations, two or more processors may be coupled via a bus to enable independent, parallel, pipelined, or multi-threaded instruction execution. Each processor may be implemented as one or more general-purpose processors, ASICs, FPGAs, GPUs, TPUs, digital signal processors (DSPs), or other suitable electronic data processing components structured to execute instructions provided by memory. The one or more processors may take the form of a single core processor, multi-core processor (e.g., a dual core processor, triple core processor, or quad core processor), microprocessor, etc. In some implementations, the one or more processors may be external to the apparatus, for example, the one or more processors may be a remote processor (e.g., a cloud-based processor). Alternatively or additionally, the one or more processors may be internal or local to the apparatus. In this regard, a given circuit or components thereof may be disposed locally (e.g., as part of a local server, a local computing system) or remotely (e.g., as part of a remote server such as a cloud based server). To that end, a “circuit” as described herein may include components that are distributed across one or more locations.

An exemplary system for implementing the overall system or portions of the implementations might include a general purpose computing devices in the form of computers, including a processing unit, a system memory, and a system bus that couples various system components including the system memory to the processing unit. Each memory device may include non-transient volatile storage media, non-volatile storage media, non-transitory storage media (e.g., one or more volatile or non-volatile memories), etc. In some implementations, the non-volatile media may take the form of ROM, flash memory (e.g., flash memory such as NAND, 3D NAND, NOR, 3D NOR), EEPROM, MRAM, magnetic storage, hard discs, optical discs, etc. In other implementations, the volatile storage media may take the form of RAM, TRAM, ZRAM, etc. Combinations of the above are also included within the scope of machine-readable media. In this regard, machine-executable instructions comprise, for example, instructions and data, which cause a general-purpose computer, special purpose computer, or special purpose processing machines to perform a certain function or group of functions. Each respective memory device may be operable to maintain or otherwise store information relating to the operations performed by one or more associated circuits, including processor instructions and related data (e.g., database components, object code components, script components), in accordance with the example implementations described herein.

It should also be noted that the term “input devices,” as described herein, may include any type of input device including, but not limited to, a keyboard, a keypad, a mouse, joystick, or other input devices performing a similar function. Comparatively, the term “output device,” as described herein, may include any type of output device including, but not limited to, a computer monitor, printer, facsimile machine, or other output devices performing a similar function.

It should be noted that although the diagrams herein may show a specific order and composition of method steps, it is understood that the order of these steps may differ from what is depicted. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative implementations. Accordingly, all such modifications are intended to be included within the scope of the present disclosure as defined in the appended claims. Such variations will depend on the machine-readable media and hardware systems chosen and on designer choice. It is understood that all such variations are within the scope of the disclosure. Likewise, software and web implementations of the present disclosure could be accomplished with standard programming techniques with rule-based logic and other logic to accomplish the various database searching steps, correlation steps, comparison steps, and decision steps.

While this specification contains many specific implementation details, these should not be construed as limitations on the scope of any inventions or of what may be claimed, but rather as descriptions of features specific to particular implementations of the systems and methods described herein. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Having now described some illustrative implementations and implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements, and features discussed only in connection with one implementation are not intended to be excluded from a similar role in other implementations.

The phraseology and terminology used herein is for the purpose of description and should not be regarded as limiting. The use of “including,” “comprising,” “having,” “containing,” “involving,” “characterized by,” “characterized in that,” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any act or element being based on any information, act, or element may include implementations where the act or element is based at least in part on any information, act, or element.

Any implementation disclosed herein may be combined with any other implementation, and references to “an implementation,” “some implementations,” “an alternate implementation,” “various implementation,” “one implementation,” or the like are not necessarily mutually exclusive and are intended to indicate that a particular feature, structure, or characteristic described in connection with the implementation may be included in at least one implementation. Such terms as used herein are not necessarily all referring to the same implementation. Any implementation may be combined with any other implementation, inclusively or exclusively, in any manner consistent with the aspects and implementations disclosed herein.

References to “or” may be construed as inclusive so that any terms described using “or” may indicate any of a single, more than one, and all of the described terms.

Where technical features in the drawings, detailed description or any claim are followed by reference signs, the reference signs have been included for the sole purpose of increasing the intelligibility of the drawings, detailed description, and claims. Accordingly, neither the reference signs nor their absence have any limiting effect on the scope of any claim elements.

The foregoing description of implementations has been presented for purposes of illustration and description. It is not intended to be exhaustive or to limit the disclosure to the precise form disclosed, and modifications and variations are possible in light of the above teachings or may be acquired from this disclosure. The implementations were chosen and described in order to explain the principals of the disclosure and its practical application to enable one skilled in the art to utilize the various implementations and with various modifications as are suited to the particular use contemplated. Other substitutions, modifications, changes, and omissions may be made in the design, operating conditions and implementation of the implementations without departing from the scope of the present disclosure as expressed in the appended claims.

What is claimed is:

1. A system, comprising:
one or more processors coupled to memory, the one or more processors configured to:
receive a chromatogram from an electropherogram device, the chromatogram having multiple signals with a time axis and an intensity axis;
generate, from the chromatogram, a plurality of base positions along the time axis, wherein each base position is generated at either a peak or inflection point in the chromatogram, and wherein multiple base positions are generated at each wide peak in the chromatogram, each wide peak having a full width half max (FWHM) greater than a predetermined width;
call, for each base position corresponding to the chromatogram, a base at the base position corresponding to a signal of the multiple signals of the chromatogram;
generate a base calling array that stores called bases, and
wherein the base calling array includes a plurality of indexes, each index in the plurality of indexes corresponds to a window along the time axis such that called bases within the window are added to the corresponding index, wherein each window partially overlaps with another window, and called bases at base positions that overlap two windows are added to both indexes.

2. The system of claim 1, wherein to generate the base calling array, the one or more processors are further configured to identify a first anchor peak and use the first anchor peak position along the time axis as a first index in the plurality of indexes.

3. The system of claim 2, wherein to generate the base calling array, the one or more processors are further configured to:
starting at a second index after the first index, for each window, identify an anchor peak if present;
if the anchor peak is present, adjust the window by a distance so that a midpoint of the anchor peak is a midpoint of the window; and
adjust subsequent windows by the distance.

4. The system of claim 1, wherein the predetermined width is greater than about 1.5X, where X is a FWHM of an anchor peak.

5. The system of claim 4, wherein generating multiple base positions at each wide peak in the chromatogram comprises generating a base position at a first predetermined position left of each wide peak and a second predetermined position right of each wide peak.

6. The system of claim 4, wherein generating multiple base positions at each wide peak in the chromatogram comprises:
identifying whether each wide peak skews left, right, or is centered;
for each wide peak that skews left, generating base positions at (⅚)X left of each wide peak and (⅙)X right of each wide peak;
for each wide peak that skews right, generating base positions at (⅚)X right of each wide peak and (⅙)X left of each wide peak; and
for each wide peak that is centered, generating base positions at ( 5/12)X right and left of each wide peak.

7. The system of claim 1, wherein the one or more processors are further configured to:
determine a number of indexes that include multiple bases; and
cause a client device to present a notification responsive to determining that the indexes with the multiple bases exceeds a threshold value.

8. The system of claim 7, wherein:
determining the number of indexes that include the multiple bases comprises determining the number of indexes including three bases and the number of indexes including four bases; and
the threshold value includes different threshold values for the number of indexes including the three bases and the number of indexes including the four bases.

9. The system of claim 1, wherein the one or more processors are further configured to:
generate a list of species from a reference database that have a DNA sequence that at least partially matches a portion of the base calling array; and
group the list of species according to Levenshtein distances between respective DNA sequences.

10. The system of claim 9, wherein grouping the list of species according to Levenshtein distances comprises grouping species together that have a percentage Levenshtein distance of 5% or less.

11. The system of claim 9, wherein the one or more processors are further configured to cause a client device to present a notification responsive to determining that more than a threshold number of groups are generated.

12. The system of claim 11, wherein the threshold number of groups is 3 groups.

13. The system of claim 1, wherein a signal of the multiple signals of the chromatogram corresponds to a base that is one of A, C, T, or G.

14. A method, comprising:
receiving a chromatogram from an electropherogram device, the chromatogram having multiple signals with a time axis and an intensity axis;
generating, from the chromatogram, a plurality of base positions along the time axis, wherein each base position is generated at either a peak or inflection point in the chromatogram, and wherein multiple base positions are generated at each wide peak in the chromatogram, each wide peak having a full width half max (FWHM) greater than a predetermined width;
calling, for each base position corresponding to the chromatogram, a base at the base position corresponding to a signal of the multiple signals of the chromatogram;
generating a base calling array that stores called bases, and
wherein the base calling array includes a plurality of indexes, each index in the plurality of indexes corresponds to a window along the time axis such that called bases within the window are added to the corresponding index, wherein each window partially overlaps with another window, and called bases generated at base positions that overlap two windows are added to both indexes.

15. The method of claim 14, wherein generating the base calling array comprises identifying a first anchor peak along the time axis and using the first anchor peak position along the time axis as a first index in the plurality of indexes.

16. The method of claim 15, wherein generating the base calling array comprises:
starting at a second index after the first index, for each window, identifying an anchor peak if present;
if the anchor peak is present, adjusting the window by a distance so that a midpoint of the anchor peak is a midpoint of the window; and
adjust subsequent windows by the distance.

17. The method of claim 14, wherein the predetermined width is greater than about 1.5X, where X is a FWHM of a standard peak.

18. The method of claim 17, wherein generating multiple base positions at each wide peak in the chromatogram comprises generating a base position at a first predetermined position left of each wide peak and a second predetermined position right of each wide peak.

19. The method of claim 17, wherein generating multiple base positions at each wide peak in the chromatogram comprises:
identifying whether each wide peak skews left, right, or is centered;
for each wide peak that skews left, generating base positions at (⅚)X left of each wide peak and (⅙)X right of each wide peak;
for each wide peak that skews right, generating base positions at (⅚)X right of each wide peak and (⅙)X left of each wide peak; and
for each wide peak that is centered, generating base positions at ( 5/12)X right and left of each wide peak.

20. The method of claim 14, further comprising:
determining a number of indexes that include multiple bases; and
causing a client device to present a notification responsive to determining that the indexes with the multiple bases exceeds a threshold value.

21. The method of claim 20, wherein:
determining the number of indexes that include the multiple bases comprises determining the number of indexes including three bases and the number of indexes including four bases; and
the threshold value includes different threshold values for the number of indexes including the three bases and the number of indexes including the four bases.

22. The method of claim 14, further comprising:
generating a list of species from a reference database that have a DNA sequence that at least partially matches a portion of the base calling array; and
grouping the list of species according to Levenshtein distances between respective DNA sequences.

23. The method of claim 22, wherein grouping the list of species according to Levenshtein distances comprises grouping species together that have a percentage Levenshtein distance of 5% or less.

24. The method of claim 22, further comprising causing a client device to present a notification responsive to determining that more than a threshold number of groups are generated.

25. The method of claim 24, wherein the threshold number of groups is 3 groups.

26. The method of claim 14, wherein a signal of the multiple signals of the chromatogram corresponds to a base that is one of A, C, T, or G.
